(19) United States
(12) Patent Application Publication
(10) Pub. No.: US 2003/0003587 A1
(43) Pub. Date: Jan. 2, 2003

(54) Molecularly imprinted polymer based sensors for the detection of narcotics

(76) Inventor: George M Murray, Columbia, MD (US)

Correspondence Address: Francis A Cooch, The Johns Hopkins University, Appied Physics Laboratory, 11100 Johns Hopkins Road, Laurel, MD, 20723-6099, US

(21) Appl. No.: 10/169,264

(22) PCT Filed: Apr. 10, 2001
(86) PCT No.: PCT/US01/11694

(51) Int. Cl. G01N 33/20; G01N 21/76

(52) U.S. Cl. 436/82; 436/164; 436/165; 436/172; 422/82.05; 422/82.06; 422/52; 422/82.07; 422/82.08; 73/655

(57) ABSTRACT

A device for measuring and detecting at least one narcotic, such as heroin and cocaine, in a fluid is provided. The device functions by selectively binding vapors of a narcotic or a narcotic present in a liquid, e.g., blood, to a luminescent molecularly imprinted polymer. The polymer possesses a securely bound luminescent lanthanide ion, such as Eu³⁺, in a coordination complex that has been templated with a narcotic.

### CROSS-REFERENCE TO RELATED APPLICATIONS

This application claims the benefit of prior filed co-pending U.S. application Ser. No. 60/195,934, filed on Apr. 10, 2000.

### BACKGROUND OF THE INVENTION

1. Field of the Invention

The present invention relates to methods for detecting a narcotic in a fluid and devices employing molecularly imprinted polymer-based sensors for detecting the narcotic in a fluid.

2. Description of the Related Art

Narcotics traffic throughout the world is a serious problem. The term “narcotics” as used herein shall be defined broadly to include illicit drugs as defined by the laws of the United States and/or by international convention and particularly includes opiates such as opium, morphine and heroin, as well as cannabinol and cocaine, as well as metabolites, salts, and pro-drugs thereof. The detection of narcotics is a concern of law enforcement officials. Since narcotics are often highly potent, quantities sufficient to provide numerous doses may be easily concealed. Even though current interdiction methodology has improved, methods and techniques for smuggling drugs have also improved, and it is a certainty that only a small percentage of narcotics entering the United States are confiscated. It is speculated that a high percentage of narcotics entering this country are concealed in enclosed spaces, such as cargo containers. On average, approximately 15 manhours are required to thoroughly search a cargo container, so that a very low percentage of the millions of cargo containers entering the United States each year are inspected. The sheer number of cargo containers and enclosed boxes of every description necessitates a method for rapid detection of narcotics.

Narcotics, as all chemicals, can be found in the body of a human. Particularly, the narcotic is typically found in the blood, urine, perspiration of a human when subjected to drug testing. Additionally, narcotics have particular vapor pressures. If vapors of a narcotic can be particularly identified within the atmosphere of a region, the presence of the narcotic may be assumed.

While chemical tests are available for substantially all narcotics to ascertain that a sample is or contains the particular narcotic, identifying their vapors is considerably more difficult. The vapor pressures of many solid substances, including most narcotics, is low and the concentration of molecules in the air will accordingly be low. Any test for vapors of a minimally volatile substance must be highly sensitive. Furthermore, such a test should be highly specific for the material to be identified, particularly when a single test is used to determine its presence. As opposed to a laboratory, where organic compounds to be identified may be isolated and subjected to a battery of tests for identification, a field test for vapors must particularly identify an unisolated compound, preferably in a single test even at low levels and in the possible presence of vapors of other similar compounds.

One reason for the low rate of drug interdiction is the lack of adequate instrumentation available to field agents. Mass spectrometers and gas chromatographs are not portable, require 100/220V power, and must be operated by well-trained technicians.

Optical sensors for the detection of analytes generally rely on small changes in the indices of refraction in response to the presence of an analyte. Of greater sensitivity and utility are sensors that respond to luminescence. Such sensors rely on the intrinsic luminescence of the analyte or in the influnence of the analyte on the luminescece of one of the sensor components. Commonly used optical sensors include planar waveguides, optical fibers, metallized prisms and diffraction gratings. These and other conventional methods typically require extensive analysis procedures that can take up to 24 hours to perform. Although all these techniques have some degree of sensitivity, they lack specificity, simplicity, rapid detection and portability.

Surface acoustic wave (SAW) devices/sensors typically comprise piezoelectric crystals that detect the mass of chemical vapors absorbed into the chemically selective coating on the sensor surface. This absorption causes a change in the resonant frequency of the sensor. An internal microcomputer measures these changes and uses them to determine the presence and concentration of chemical agents. Conventional SAW sensors have coatings that exhibit unique physical properties that allow a reversible absorption of an analyte, such as chemical vapors. The polymer-coated sensor combined with trainable software loaded into a microcomputer to recognize chemical vapor signature patterns, completes the analysis. Although conventionally available SAW sensors meet the needs of real time analysis and offer the additional benefits of multiple gas detection capability, rugged designs, computerized control, easy operation and low cost, they typically lack selectivity, especially with respect to chemically similar compounds thus making false positive readings a major concern.

It is therefore necessary to develop narcotics detection devices and methods that address the above and other problems. Therefore, instrumentation is needed that is readily portable, operable with a minimum of training, capable of detecting narcotics at extremely low concentration, minimally affected by interfering substances, rugged, and completely user friendly.

### SUMMARY OF THE INVENTION

It is a primary object of the present invention to provide a method of detecting the presence of vapors of a narcotic within a region.

It is also a primary object of the present invention to provide a method of detecting the presence of a narcotic in a liquid.

It is a further object of this invention to employ a molecularly imprinted polymer which has been templated with one or more narcotics for the detection of a narcotic in a fluid.

Yet a further object of the present invention is to provide a sensor that is capable of detecting a narcotic in a fluid in parts per billion (ppb) to parts per quadrillion (ppq) levels, which is faster than other similar sensors and that is free from false positive results.

Another object of the present invention is to provide an optical based sensor device for the remote detection of narcotics in a fluid.

It also an object of the present invention to provide a SAW device for detecting the presence of narcotics in a fluid.

A still further object of the present invention is to provide luminescent materials containing lanthanide-complexes having a relatively long-lived luminescence or phosphorescence.

The above and other objects are met by a spectroscopic sensor/probe comprising a lanthanide-complex bound to a molecularly imprinted polymer. By “complex” it is meant a coordination compound formed by the union of a lanthanide ion with a non-metallic ion or molecule called a ligand or complexing agent. The lanthanide-complexes of the present invention comprise at least one lanthanide ion and at least one ligand.

The molecularly imprinted polymer may be bound to a suitable substrate, such as, for example, a badge worn by a person or animal, for the detection of narcotics vapors. When the person or animal wearing the badge enters a zone or environment containing narcotic vapors, the narcotic vapor(s) bind(s) to the lanthanide-complex in the molecularly imprinted polymer thereby causing the lanthanide-complex to luminesce.

The molecularly imprinted polymer may also be bound to a suitable substrate such that when a person, e.g., a law enforcement official, exposes at least a portion of the substrate containing the polymer to an environment containing at least a portion of a liquid, e.g., blood, of an individual which contains at least one narcotic therein, the narcotic(s) bind(s) to the lanthanide-complex in the molecularly imprinted polymer thereby causing the lanthanide-complex to luminesce.

In addition, molecularly imprinted polymer of the present invention may be modified for use in the extraction and preconcentration of the ions, such as lead, prior to analysis. This is analogous to the process of using solid phase micro-extraction to preconcentrate organic compounds prior to chromatographic analysis. In this case, the chromatographic separation could be eliminated and the sample quantitated by a chromatographic detector, such as mass spectrometer.

In still another embodiment, the present invention is directed to a fiber optic or other waveguide sensor device for detecting the presence of a narcotic, such as opium, morphine, heroin, cannabinol and cocaine, as well as metabolites, salts, degradants (as in the case of methylbenzoate from cocaine) and pro-drugs thereof in a fluid, the sensor comprising:

at least one optical fiber means having a proximal end and a distal end for transmitting light energy, the proximal end being disposed within a probe housing,

a molecularly imprinted polymer containing a lanthanide-complex disposed on or bonded to the distal end of the optical fiber means, wherein the lanthanide-complex is capable of chemically binding with said narcotic,

light source means for generating excitation energy, said light source means operatively associated with said optical fiber means such that said excitation light passes through said optical fiber means, and

detection means operatively associated with said optical fiber means, for detecting an emission signal generated by said lanthanide complex. As used herein, the term “light” refers to optical radiation, whether ultraviolet, visible or infrared. FIG. 1 depicts a sensor device having the features of this embodiment.

In another embodiment, the present invention is directed to a surface acoustic wave sensor for detecting the presence of a narcotic in a fluid said sensor having been adapted to comprise a molecularly imprinted polymer having a lanthanide-complex bound thereto. In particular, the surface acoustic wave sensor of the present invention comprises:

a film of a molecularly imprinted polymer containing a lanthanide-complex disposed on a substrate such as alumina or a piezocrystal substance such as quartz crystal, wherein the lanthanide-complex is capable of chemically binding with fluids containing a narcotic;

input and output transducers disposed on the film or substrate; and

a function generator operatively associated with the input transducer for generating a surface acoustic wave along a delay line. FIG. 2 depicts a sensor device having the features of this embodiment.

Additional aspects, embodiments and advantages of the present invention will be set forth, in part, in the description that follows, or may be learned from practicing or using the present invention. The objects and advantages may be realized and attained by means of the features and combinations particularly pointed out throughout this description and the appended claims. It is to be understood that the foregoing general description and the following detailed description are exemplary and explanatory only and are not to be viewed as being restrictive of the invention as claimed.

The term “fluid” is used herein in its art-recognized sense, i.e., as referring to include gases such as vapors and liquids such as, for example, semen, blood, urine, perspiration, etc.

### BRIEF DESCRIPTION OF THE DRAWINGS

Other objects, features and advantages of the present invention will occur to those skilled in the art from the following description of preferred embodiments and the accompanying drawings, in which:

FIG. 1 is a schematic drawing of an optical sensor of the present invention.

FIG. 2 is a schematic drawing of a SAW sensor of the present invention.

FIG. 3 is a schematic representation of molecular imprinting to obtain a molecularly imprinted polymer of the present invention.

FIG. 4 depicts structural representations of ligand monomers that may used in accordance with the principles of the present invention.

FIG. 5 is laser excited luminescence spectra of complexes with dibenzoylmethane.

FIG. 6 is laser excited luminescence spectra of the 1) tris complex, 2) the complex with sodium benzoate added, 3) the sodium benzoate complex in a polymer and coated on a fiber and 4) the spectrum of the complex with pyridinium benzoate added

FIGS. 7 and 8 shows describe the silanization procedure.

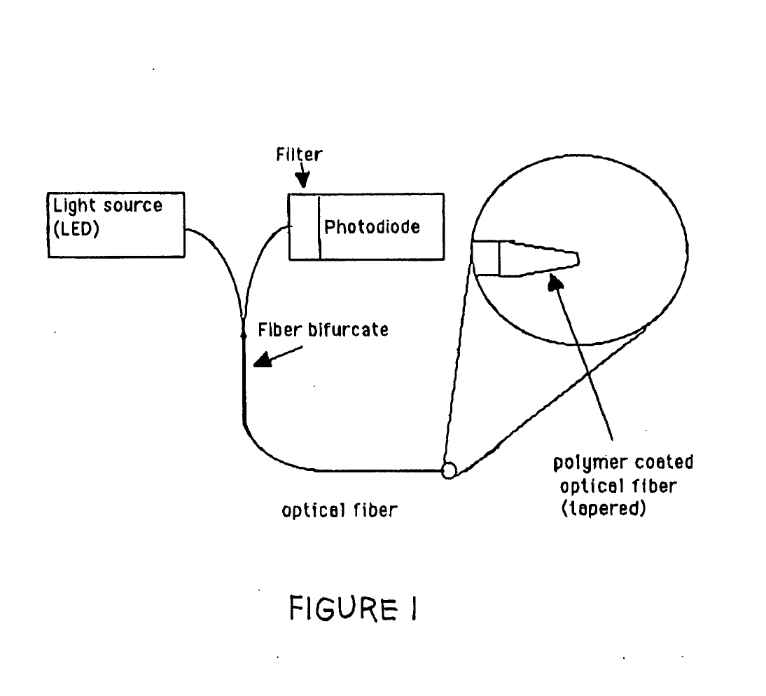

### DESCRIPTION OF THE PREFERRED EMBODIMENTS

References cited throughout this written description are incorporated by reference herein in their entirety to more fully describe the state of the art to which they pertain.

It will be appreciated that the process steps and structures described below do not form a complete process flow for manufacturing devices encompassed within the appended claims. The present invention can be practiced in conjunction with conventionally known sensor manufacturing, and only so much of the commonly practiced manufacturing processes are included as is necessary for an understanding of the present invention.

The present invention combines the techniques of molecularly imprinting and sensitized lanthanide luminescence, thereby providing multiple criteria for selectivity for an analyte constituting a narcotic and virtually eliminating the possibility for false positive readings. The lanthanide elements, also known as the rare earth elements, consist of the elements having atomic numbers from 57 to 71. As used herein, the term “lanthanide” refers to the following elements of the periodic table: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). In the present invention, a lanthanide is chosen as the tranducer because the trivalent lanthanide ions have excellent spectroscopic properties such as long luminescence lifetimes and narrow bandwidths, usually only a few nanometers. Preferred lanthanide ions that exhibit a narrow-line luminescence and include the +3 ions of samarium, europium, dysprosium, terbium, and neodymium, with europium and terbium being most preferred.

As used herein, the terms “molecularly imprinted molecule,” “molecularly imprinted polymer” and “MIP” refer to a molecular mold-like structure that has preorganized interactive moieties complementing the spacing of binding sites on a narcotic molecule. The interactive moieties can be, for example, chemical groups or affinity ligands. The geometrical organization of interactive moieties imparts selective binding characteristics for the narcotic molecule onto the imprinted polymer. The term “selective binding interactions” is intended to refer to preferential and reversible binding exhibited by an imprinted polymer for its template molecule (herein, the narcotic) compared to other non-template molecules. Selective binding includes both affinity and specificity of the imprinted polymer for its template molecule.

The origins of molecularly imprinted molecules trace back to the notion of Linus Pauling that the body assembled a new protein complement (i.e., an antibody) by using the foreign intruder as a template. Although it was later determined that this is not how antibodies are selected in vivo, this template concept stimulated significant thought and research. Molecular imprinting creates specific recognition sites in materials, such as polymeric organic materials. Known molecular imprinting techniques involve crosslinking materials in the presence of a functional monomer or mixture of monomers. The template molecule interacts with a complementary portion of a functional monomer, either covalently or by other interactions such as ionic, hydrophobic or hydrogen bonding, so that recognition sites for the template molecule can be provided in the substrate material. The template molecule is then removed from the substrate to leave a “cavity” or recognition site. Linus Pauling reasoned that shape specificity was obtained by using a target antigen to arrange the complementary shape of an antibody. Thus, a nonspecific molecule can be shaped to the contours of a specific target, and when the target is removed, the shape is maintained to give the antibody a propensity to rebind the antigen. This process is known as “molecular imprinting” or “templating.”

The target or template molecule directs the positioning of the encapsulating antibody by the interactions that occur between certain sites on the target and complementary sites on the antibody. The sites that allow complementary associations are certain arrangements of atoms that exhibit an electrostatic attraction of a specific kind. These localized atomic arrangements are sometimes referred to as “functional groups.” The functional groups on a molecule help to define the molecule's overall chemical properties. In general, the MIP should exhibit as closely as possible the reverse topology of the template molecule. For example, if the template molecule has an cationic group at a specific location, then the MIP should have a anionic group at that location.

The synthetic production of polymers with selective binding for a specific cation is achieved by providing polymers with cavities lined with complexing groups or “ligands” arranged to match the charge, coordination number, coordination geometry, and size of the target cation. Anion complexing polymers are made in a similar manner, but typically employ a trapped metal ion that has a large affinity for the anion in question. These cavity-containing polymers are produced by using a specific ion as a template around which monomeric complexing ligands will be self-assembled and later polymerized. The complexing ligands are ones containing functional groups known to form stable complexes with the specific ion and less stable complexes with other ions.

When lanthanide ions are chelated with appropriate ligands, a significant enhancement of the luminescence intensity is obtained. The chelated lanthanide complexes of the present invention provide a sensitive means of analysis with low limits of detection when incorporated in a MIP. For example, lanthanide ions can form complexes with various organic molecules such as beta-diketones, polyaminopolycarboxylic acids (EDTA and the like), (poly)pyridines and calixarenes. Moreover, ligands containing organic chromophores possessing suitable photophysical properties provide highly luminescent lanthanide complexes. See, Jenkins, A. L., Uy, O. M. and Murray, G. M., “Ultratrace Determination of Selected Lanthanides by Luminescence Enhancement,” Anal. Chem., 68(17):2974-2980 (1996) (the entire disclosure of which is incorporated herein by reference). With a careful selection of complexing ligands, metal complexes can be synthesized by mixing stoichiometric amounts of a lanthanide metal salt and the complexing ligand in an aqueous solution and evaporating to near dryness. Water or alcohol/water mixtures of the lanthanide metal and ligand in stoichiometric ratios, evaporated to dryness, are preferred to obtain near quantitative yield of the desired complex compound. To make complexes that contain target anions, it is preferred to make mixed ligand complexes that have a one-to-one stoichiometric ratio of target anion to complex. This can be accomplished by synthesizing lanthanide metal ion complexes with the proper coordination number of tightly binding ligands such that a single target analyte could bind by replacing a very weakly bound substituent.

A MIP in accordance with the principles of the present invention can be prepared using known methods. The polymerization reaction mixture for the preparation of the MIP constitutes a narcotic, polymerizable functional monomers, which include an effective amount of one or more crosslinking agents to provide a sufficiently rigid structure, inert solvent, and a free radical or other appropriate initiator. Mixtures of monomers and crosslinking agents can be used in the polymerization method.

In general, two approaches to the production of a molecularly imprinted polymer have been developed, and either can be used in the method disclosed herein. In the first method, a narcotic molecule is covalently bound to a polymerizable monomer, and after polymerization, the covalent bond is cleaved to release the narcotic molecule from the polymeric mold. Using this method, a selected narcotic molecule is attached to a polymerizable moiety using any appropriate method. The polymerizable narcotic molecule should contain a linkage that can be broken to release the narcotic after the MIP is formed, without adversely affecting the MIP. The bond that is cleaved to release the narcotic molecule can optionally provide an additional polar or ionic site for design and imprinting of the mimic. In the second and more preferred method, polymerizable monomers arrange themselves about a narcotic molecule based on noncovalent interactions (such as ionic, hydrophobic, steric, electrostatic, and hydrogen bonding interactions), and after polymerization, the non-covalently bound narcotic molecule is simply leached or washed out.

For example, a MIP in accordance with the principles of the present invention may be formed by:

mixing a narcotic molecule and polymerizable imprint monomers containing a chelated lanthanide under conditions whereby the imprint monomers covalently or noncovalently bind the narcotic molecule through interactions with the chelated lanthanide;

forming the MIP from the monomers by adding a cross-linking agent to the mixture that produces covalent bonds between ligands on adjacent monomers; and

removing the narcotic molecule from the MIP.

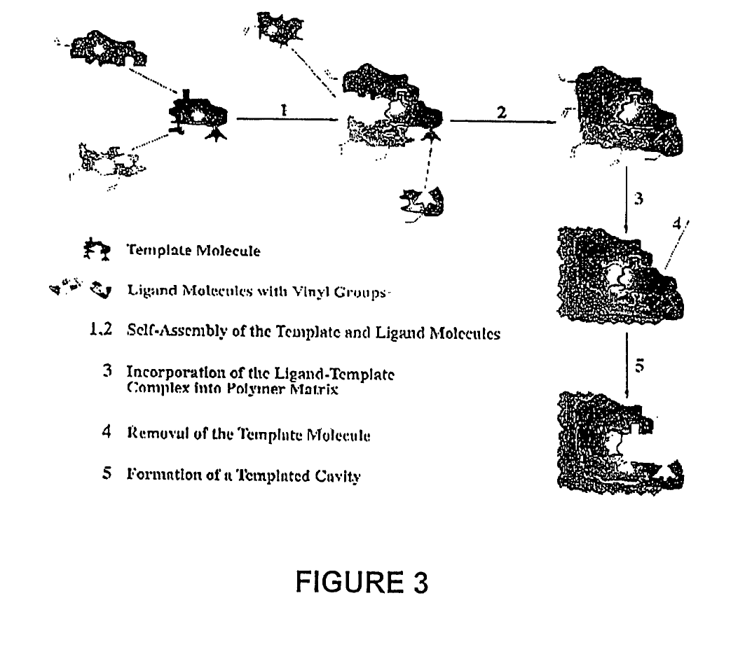

The resultant MIP will covalently or noncovalently bind the narcotic molecule with which it was imprinted with higher affinity than other, similar, though not identical, species. FIG. 3 is a schematic representation of molecular imprinting showing self assemble of narcotics and ligand molecules (1, 2); incorporation of the ligand-narcotic complex into the polymer matrix (3); removal of the narcotic molecule; and formation of the templated cavity (5). Other methods for preparing MIPs are described in U.S. Pat. Nos. 5,110,883; 5,321,102; 5,372,719; 5,310,648; 5,208,155; 5,015,576; 4,935,365; 4,960,762; 4,532,232; 4,415,655; and 4,406,792, the entire disclosures of which are incorporated by reference herein.

It will be appreciated that a key step in making a molecularly imprinted polymer is to form a complex that will survive the polymerization process and leave behind a suitable set of binding sites when the narcotic molecule is removed. To form such a complex, ligands must be chosen that exhibit sufficiently large affinities to resist dissociation. The success of the end product hinges on the selection of the ligating monomer. In addition, the polymerization process must provide sufficient rigidity to effect structural “memory” but be sufficiently flexible to allow removal of the narcotic molecule.

Virtually any narcotic molecule can be employed in the practice of the present invention. Examples of such narcotic molecules include, but are not limited to, amphetamines, methamphetamine, barbiturates, benzodiazepine, buprenorphine, cannabinoids, cocaine, fentanyl, LSD, methadone, nicotine, opiates such as morphine and heroin, and phencyclidine, as well as metabolites, salts, degradants (as in the case of methylbenzoate from cocaine) and pro-drugs thereof.

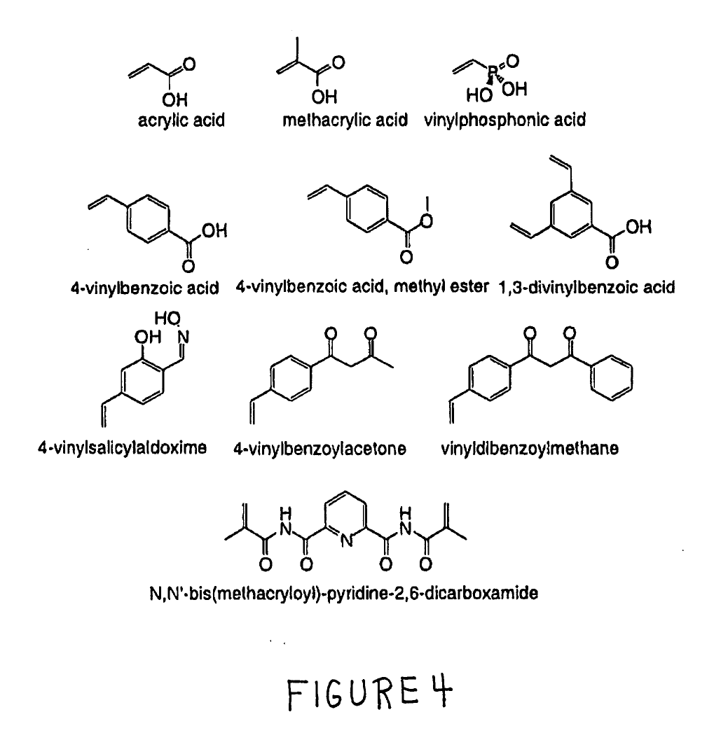

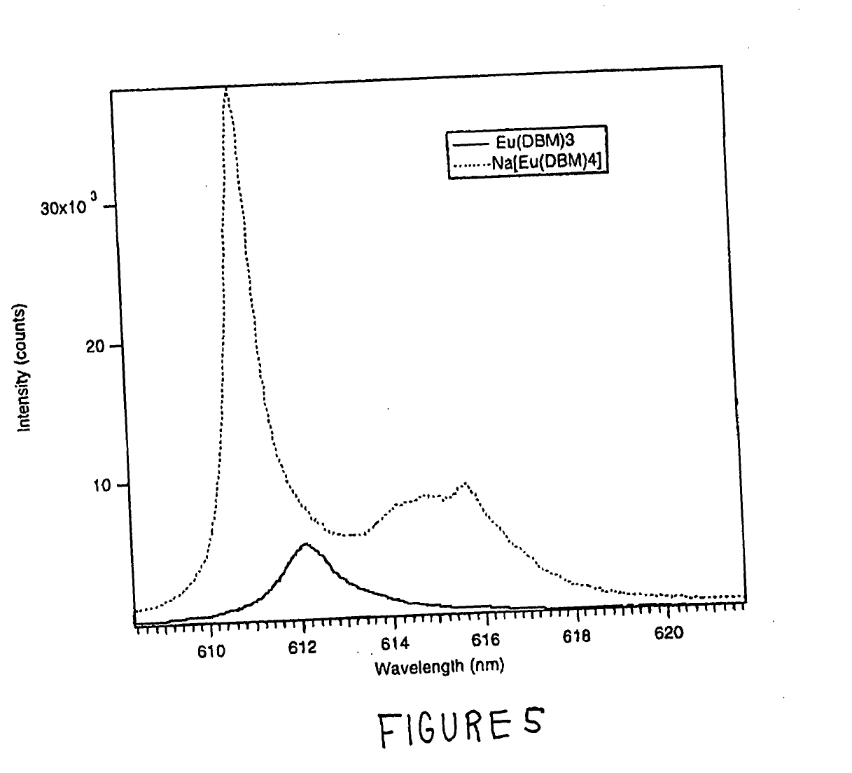

Any suitable monomer that provides an accurate imprint of the template molecule on polymerization may be used for synthesizing a MIP in accordance with the principles of the present invention. For example, structural representations of preferred monomers are depicted in FIG. 4, namely (a) acrylic acid, (b) methacrylic acid, (c) vinylphosphonic acid, (d) 4-vinylbenzoic acid, (e) 4-vinylbenzoic acid, methyl ester, (f) 1,3-divinylbenzoic acid, (g) 4-vinylsalicylaldoxime, (h) 4-vinylbenzoylacetone, (i) vinyldibenzoylmethane, and (j) N,N′-bis(methacryloyl)-pyridine-2,6-dicarboxamide. Other examples include 2-hydroxy-1,2-di-4-vinylphenylethanone (benzoin oxime vinyl derivative), 4-vinyl-2-hydroxybenzaldehyde oxime (vinylsalicylaldoxime) and methyl-3,5-divinyl benzoate (MDVB). Further examples of other suitable monomers for use in either of the two approaches discussed above, include, but are not limited to, those described in the references cited in this written description and the Examples provide below. Further suitable nonlimiting examples of monomers that can be used for preparing a MIP of the present invention include: methylmethacrylate, other alkyl methacrylates, alkylacrylates, ally or aryl acrylates and methacrylates, cyanoacrylate, styrene, alpha.-methyl styrene, vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy)ethyl methacrylate 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; acryloyl chloride; (R)-.alpha.-acryloxy-.beta.,.beta.′-dimethyl-g-butyrolactone; N-acryloxy succinimide N-acryloxytri.s(hydroxymethyl) aminomethane; N-acryloly chloride; N-acryloyl pyrrolidinone; N-acryloyl-tris(hydroxymethyl)amino methane; 2-amino ethyl methacrylate; N-(3-aminopropyl)methacrylamide; (o, m, or p)-amino-styrene; t-amyl methacrylate; 2-(1-aziridinyl)ethyl methacrylate; 2,2′-azobis-(2-amidinopropane); 2,2′-azobisisobutyronitrile; 4,4′-azobis-(4-cyanovaleric acid); 1,1′-azobis-(cyclohexanecarbonitrile); 2,2-azobis-(2,4-dimethylvaleronitrile); 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; .beta.-bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; N-t-butylacrylamide; butyl acrylate; butyl methacrylate; (o,m,p)-bromostyrene; t-butyl acrylate; (R)-carvone; (S)-carvone; (−)-carvyl acetate; cis 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstryene; 3,4-dimethylstryene; divinyl benzene; 1,3-divinyltetramethyl disiloxane; 8,13-divinyl-3,7,12,17-tetramethyl-21H,23H-porphine; 8,1 3-divinyl-3,7,12,17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethyl-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; lead (II) acrylate; (±)-:linalool; linalyl acetate; p-mentha-1,8-diene; p-mentha-6,8-dien-2-ol; methyleneamino acetonitrile; methacrolein; [3-(methacryloylamino)-propyl]trimethylammonium chloride; methacrylamide; methacrylic acid; methacrylic anhydride; methacrylonitrile; methacryloyl chloride; 2-(methacryloyloxy)ethyl acetoacetate; (3-methacryloxypropyl)trimethoxy silane; 2-(methacryloxy)ethyl trimethyl ammonium methylsulfate; 2-methoxy propene (isopropenyl methyl ether); methyl-2-(bromomethyl)acrylate; 5-methyl-5-hexen-2-one; methyl methacrylate; N,N′-methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; alpha.-methyl styrene; t-a-methylstyrene; t-.beta.-methylstyrene; 3-methylstyrene; methyl vinyl ether; methyl vinyl ketone; methyl-2-vinyloxirane; 4-methylstyrene; methyl vinyl sulfonee; 4-methyl5-vinylthiazole; myrcene; t-.beta.-nitrostyrene; 3-nitrostyrene; 1-nonadecene; 1,8-nonadiene; 1-octadecene; 1,7-octadiene; 7-octene-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyano ethylene; tetramethyldivinyl siloxane; trans 3-chloroacrylic acid; 2-trifluoromethyl propene; 2-(trifluoromethyl)propenoic acid; 2,4,4′-trimethyl-1-pentene; 3,5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethyl sulfone; 4-(vinyl benzyl)-2-chloroethyl sulfone; N-(p-vinyl benzyl)-N,N′-dimethyl amine; 4-vinyl biphenyl (4-phenyl styrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl cyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinyl imidizole; vinyl iodide; vinyl laurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinyl quinoline; 1-vinyl silatrane; vinyl sulfone; vinyl sulfone (divinylsulfone); vinyl sulfonic acid sodium salt; o-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy)silane; vinyl 2-valerate and the like.

Acrylate-terminated or otherwise unsaturated urethanes, carbonates, and epoxies can also be used in the MIP. An example of an unsaturated carbonate is allyl diglycol carbonate (CR-39). Unsaturated epoxies include, but are not limited to, glycidyl acrylate, glycidyl methacrylate, allyl glycidyl ether, and 1,2-epoxy-3-allyl propane.

Crosslinking agents that lend rigidity to the MIP are known to those skilled in the art, and include di-, tri- and tetrafunctional acrylates or methacrylates, divinylbenzene (DVB), alkylene glycol and polyalkylene glycol diacrylates and methacrylates, including ethylene glycol dimethacrylate (EGDMA) and ethylene glycol diacrylate, vinyl or allyl acrylates or methacrylates, divinylbenzene, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or bismethacrylamide, including hexamethylene bisacrylamide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methy:L-2-isocyanatoethyl methacrylate, 1,1-dimethyl-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, and the like.

Any ratio of simple monomers to crosslinking monomers can be used that provides a structure of appropriate integrity. Those skilled in the art can select suitable ratios of monomers to provide the desired structural integrity.

While free radical polymerization is preferred, monomers can also be selected that are polymerized cationically or anionically. Polymerization conditions should be selected that do not adversely affect the narcotic molecule. Any UV or thermal free radical initiator known to those skilled in the art for free radical polymerization can be used to initiate this method. Examples of UV and thermal initiators include benzoyl peroxide, acetyl peroxide, lauryl peroxide, azobisisobutyronitrile (AIBN), t-butyl peracetate, cumyl peroxide, t-butyl peroxide, t-butyl hydroperoxide, bis(isopropyl)peroxy-dicarbonate, benzoin methyl ether, 2,2′-azobis(2,4-dimethylvaleronitrile), tertiarybutyl peroctoate, phthalic peroxide, diethoxyacetophenone, and tertiarybutyl peroxypivalate, diethoxyacetophenone, 1-hydroxycyclohexyl phenyl ketone, 2,2-dimethyoxy-2-phenyl-acetophenone, and phenothiazine, and diisopropylxanthogen disulfide.

When polymerization is complete, the crosslinked polymer may be washed, cryogenically ground to a uniformly fine powder, and extensively eluted with nonpolar solvents to remove unreacted complex. The steps of grinding and/or freezing in liquid nitrogen may be used to maximize surface area and allow for access by the various reagents and samples. Freezing allows the polymer to become brittle enough to be ground and prevents distortions of the polymer by the heat of friction. Polymers used in the construction of optical sensors may be prepared in situ on the distal end of an optical fiber whose surface is prepared by binding a polymerizable agent on the surface.

After polymerization, the narcotic molecule may be removed in a manner that does not adversely affect the imprinted cavity. If the narcotic molecule is covalently bound, it is removed using the mildest conditions possible for the cleavage of the covalent bond. To accomplish this, acetone or other suitable organic solvent may be used to swell the resultant polymers, allowing greater access to the coordinated metal ions because templated polymers have a relatively low amount of functionalization and are primarily nonionic matrices. Subsequent to the removal of unreacted monomer, a 1 N aqueous acidic solution may be mixed into the acetone washes, with increasing aqueous acidic phase in each sequential wash, to remove the narcotic molecules from the cavities. Preferably an acidic solvent is used having a pH of about 4.5 or less.

As one skilled in the art can readily appreciate, the preferred synthetic schemes and embodiments described above and in the Examples below are not intended to comprise a comprehensive list of all means by which the MIPs described and claimed herein may be synthesized. It will be understood that the specified materials and conditions are important in practicing the invention but that unspecified materials and conditions are not excluded so long as they do not prevent the benefits of the invention from being realized. Other suitable methods and starting materials will be evident to those having skill in the art. Additionally, the various synthetic steps described throughout this written description may be performed in an alternate sequence or order to obtain the present invention.

In a preferred embodiment, MIP may be bound to a suitable substrate, such as, for example, a dosimeter-like badge worn by a person or animal, for the detection of narcotic vapors. When the person or animal wearing the badge enters an area or environment containing vapors of a narcotic, the narcotic vapors bind to the lanthanide-complex in the molecularly imprinted polymer thereby causing the lanthanide-complex to luminesce.

In another preferred embodiment, MIP may be bound to a suitable substrate for the detection of at least one narcotic in a liquid, e.g., semen, blood, urine or perspiration. When the person or animal holding the suitable substrate exposes at least a portion of the substrate containing the MIP to an environment containing at least a portion of the liquid, the narcotic binds to the lanthanide-complex in the molecularly imprinted polymer thereby causing the lanthanide-complex to luminesce.

In yet another preferred embodiment, the present invention is directed to a fiber optic sensor device for detecting the presence of a narcotic in a fluid, the sensor comprising:

at least one optical fiber means having a proximal end and a distal end for transmitting light energy, the proximal end being disposed within a probe housing,

a molecularly imprinted polymer containing a lanthanide-complex disposed on, or bonded to, the distal end of the optical fiber means, wherein the lanthanide-complex is capable of chemically binding with said narcotic,

light source means for generating excitation energy, said light source means operatively associated with said optical fiber means such that said excitation light passes through said optical fiber means, and

detection means operatively associated with said optical fiber means, for detecting an emission signal generated by said lanthanide complex.

Suitable non-limiting examples of light source means include an argon laser, blue laser, tunable laser, light emitting diode (LED), and the like.

Suitable non-limiting examples of detection means include a spectrophotometer, spectrometer (gas or mass), photomultiplier tube, monochromator equipped with a CCD camera, filters, the naked eye, and the like.

In this embodiment, the portable device may employ a modulated laser diode for excitation and a small photosensor module for detection, with the output going to a microprocessor controlled grated integrator. In addition, an optical multiplex switch may be incorporated into the design so that many sensors can be coupled to one control system, which will allow monitoring of a large area such as found in a building, subway station, shopping mall, airport, etc.

In use, vapors of a narcotic or a liquid containing a narcotic, if present, bind to the lanthanide-complex in the molecularly imprinted polymer causing it to luminesce. Light from the light source means travels along the optical fiber to its distal end where it undergoes a change caused by interaction with the lanthanide-complex. The modified light returns along the same or another fiber to the detection means which interprets the returned light signal. Detection is based on the change that occurs in the lanthanide's luminescence spectrum when vapor of a narcotic binds to the lanthanide-complex.

Optionally, the distal end (working end) of the sensor may be enclosed within a semi-permeable membrane to separate the narcotic containing media being analyzed from the probe. One function of the membrane is to separate, as far as possible, the narcotic (i.e., those components in a sample that can bind to the probe) from interferents (i.e., compounds which may be present but are undesirable because they either interfere with the progress of the desired determination reactions or take part in reactions of their own which compete with those of the narcotic whose detection is being sought and distort or overwhelm the signals that are to be measured).

In yet another preferred embodiment, the present invention is directed to a SAW sensor for detecting the presence of narcotic vapors. The SAW sensor comprises a molecularly imprinted polymer having a lanthanide-complex bound thereto. In particular, the surface acoustic wave sensor of the present invention, comprises:

a film of a molecularly imprinted polymer containing a lanthanide-complex disposed on a substrate such as alumina or a piezocrystal substance such as quartz crystal, wherein the lanthanide-complex is capable of chemically binding with vapors of a narcotic;

input and output transducers disposed on the film or substrate; and

a function generator operatively associated with the input transducer for generating a surface acoustic wave along a delay line.

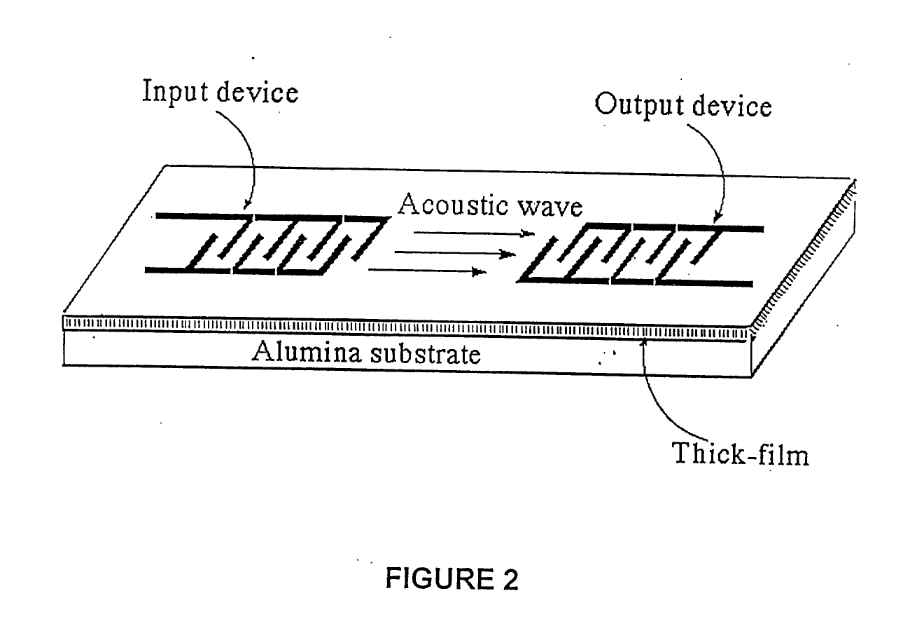

FIG. 2 depicts a SAW device in accordance with the present invention. In use, the function generator supplies a pulse modulated sine signal to the input transducer. The generated surface acoustic wave is modulated in the same way as the input electrical signal. The acoustic energy is converted again into an electric signal in the output transducer which may be connected to a microcomputer. This signal brings information about the amplitude, phase, frequency and velocity of propagation of the surface acoustic wave on the film. When narcotics vapors bind to the lanthanide-complex, the sensor substrate will perturb the surface acoustic wave propagation along a so called delay line, which is then detected using conventional means, such as an LED, microcomputer, etc.

### EXAMPLES

The present invention will be further illustrated in the following, non-limiting Examples. The Examples, which exemplify a fiber optic sensor for detecting vapors of methylbenzoate are illustrative only and do not limit the claimed invention regarding the materials, conditions, process parameters and the like recited herein.

The sensor described below was designed to measure the metabolite of cocaine degradant methylbenzoate that has been identified by law enforcement to be the chemical detected by drug tracking dogs associated with illicit cocaine.

Unless otherwise indicated, the reagent materials were obtained from commercial suppliers and used without further purification. Analytical reagent grade chemicals were used along with deionized water to prepare solutions. The reagents used in the synthesis of the vinyl substituted ligands such as: 4-bromo-benzoic acid methyl ester, 1,3-Bis-(4-bromo-phenyl)-propane-1,3-dione, Bu₃(vinyl)Sn, BHT, Pd(PPh₃)₄, sodium hydride, were obtained form Aldrich (Milwaukee, Wis.). Solvents such as dimethoxyethane, toluene, acetone, methanol, and ethanol were obtained from Fisher Scientific (Pittsburgh, Pa.).

Instrumentation. Luminescence was excited using an Onmichrome Model 543-AP-401 Argon Ion Laser (Omnichrome, Chino, Calif.) or by a blue light emitting diode or LED (World Precision Instruments, Sarasota, Fla.). Spectra were collected using an f/4, 0.5 m monochromator (Chromex, Albuquerque, N. Mex.) equipped with a Model ST-6 CCD (Santa Barbara Instruments Group, Santa Barbara, Calif.) using Kestrel Spec Software (Rhea Corporation, Wilmington, Del.). Molecular absorbance spectra were obtained using a Cary 50 UV/VIS spectrophotometer (Varian, Victoria, Australia). Radiative lifetimes and quantum efficiencies were measured using a Quanta Master Spectrophosphorimeter (Photon Technologies Inc., Ontario, Canada). Electron micrographs were obtained using a Topcon DS-701 dual stage scanning electron microscope (SEM) (Topcon, Paramus, N.J. 07652). Metal concentrations were determined using a Hewlett Packard 4500 Series ICP-MS model G1820A (Hewlett Packard, Wilmington, Del. 19808). Graphs and spectra were plotted and calculations performed using Igor Pro Software (WaveMetrics Inc., Lake Oswego, Oreg. 97035).

### Example 1

### Compound Preparation

Imprinting complexes employing Eu³⁺ as the optical transducer were synthesized in the following manner. Vinyl substituted bidentate chelating β-diketone ligands were used to form tris complexes of Eu³⁺. Benzoate anion was used to template for methyl benzoate since the anion allowed better control of complex stoichiometry. The resultant complex was redissolved in ethanol and an ethanolic solution containing an equivalent of vinylpyridinium benzoate was added dropwise. The use of an organic counter-cation improves complex solubility in the polymer and by using a vinyl-substituted counter-cation the complex is held more strongly in the polymeric matrix. The mixture was refluxed for an hour. A yellow precipitate formed upon cooling. The precipitate was filtered, washed with cold ethanol and dried in a desiccator. The compound was analyzed for Eu content by ICP-MS. The luminescence spectra of the compound were recorded by using both an Ar Ion Laser at 465.8 nm and a blue LED.

### Example 2

### Polymer Preparation

Styrenic block copolymers were prepared and the optimal mole percent complex for the preparation of the polymer coating determined. Polymers were prepared by dissolving 1 to 5 mole percent complex compound in 89-93 mole percent styrene and 5 mole percent divinylbenzene as a crosslinker. Approximately 1 mole percent of azobisisobutylnitrile (AIBN) was added as an initiator to the mixture described in Example 1. The resulting solutions were placed in glass vials, purged with nitrogen, and sealed using Parafilm and screw on tops. The resulting translucent polymers were bright yellow in color indicative of the enolate form of the β-diketone and upon excitation with a UV lamp, displaying the characteristic red-orange luminescence of europium. The luminescence of the benzoate containing polymers was easily discernable to be different form the polymers that did not contain benzoate (tris chelate) or those containing an additional equivalent of β-diketone (tetrakis chelate). Lower percent complex polymers displayed weaker luminescence characteristics and higher percent polymers gave higher intensity. Polymers with greater than 5 mole percent complex were not used since the complex solubility was exceeded.

The monomer solutions were sonicated for 2-4 hours at 60° C. (Sonication is believed to help maintain homogeneity in the polymer.) Zeng, X.; Murray, G. M. Separation Science and Technology, 31:2403-2418 (1996). After sonication, the partially polymerized material was placed in an oil bath at 60° C. and allowed to cure overnight. The resulting block copolymers were ground to expose a larger surface area of the polymer and facilitate the removal of the imprinting ion. Once ground, the template ion is removed in two steps (Id.): (1) swelling in water and gradually increasing amounts of methanol (Helferich, F., Ion Exchange; McGraw-Hill: New York, 511(1962)) to remove unreacted monomer and expand the polymer pores, (this produces accessible sites and facilitates the removal of the imprinting ion, and (2) removal of the imprinting ion by acid washing. Acid washing (1.0 mM HCL) facilitates the removal of benzoate.

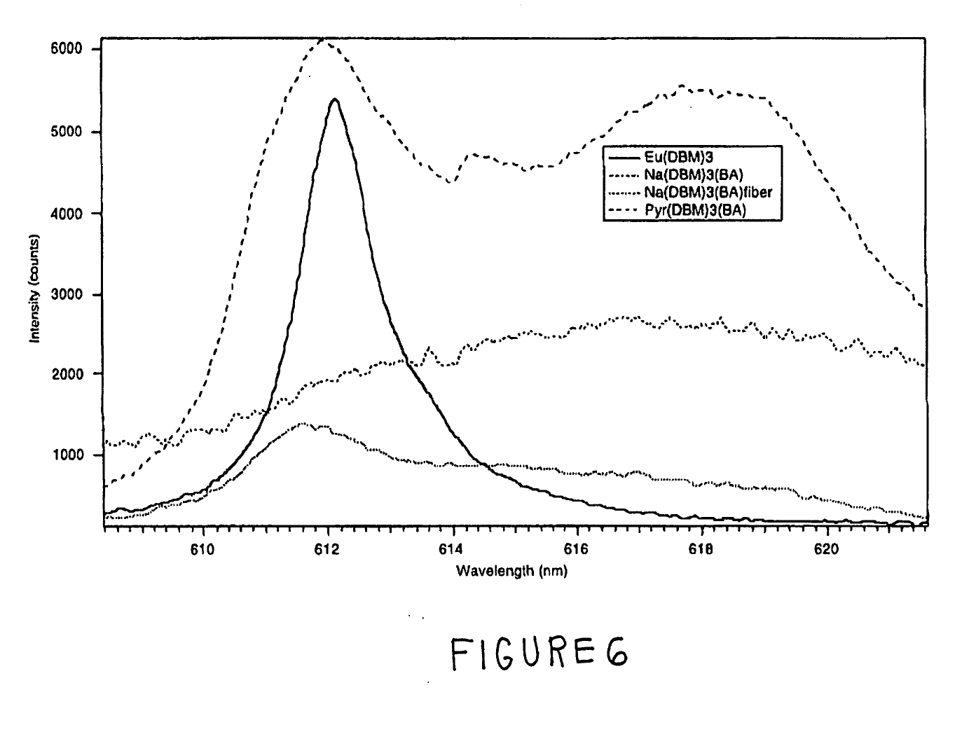

The optimal conditions for swelling the polymer include a series methanol water washes, followed by washing with a weak hydrochloric acid solution. The spectrum of the washed polymer shows a reduction in the luminescence of the Eu band associated with benzoate, demonstrating that benzoate was effectively removed. The overall intensity of the polymer's luminescence also decreases upon washing. The washed polymer was tested for its ability to rebind benzoate by exposing it to 100 ppm solution of sodium benzoate. The bezoate influenced band emission circa 617 nm was observed to increase. See FIG. 6.

### Example 3

### Fiber Optic Sensor

A fiber optic sensor comprising a 400 micron optical fiber (Thor Labs, Newton, N.J., 07860) with the polymeric sensing element chemically bound on its distal end was constructed. The fibers were prepared by terminating one end with an SMA connector and removing the cladding from and polishing the distal end using the procedures outlined in the “Thor Labs Guide to Connectorization and Polishing of Optical Fibers”. The tips were dipped into the chemically initiated viscous copolymer described in Example 2 leaving a uniform layer on the fiber. The polymer finished curing under a UV illuminator, overnight. Coated fibers were conditioned in a manner similar to the ground polymers as outlined above. Final versions of the sensor were prepared using a tapered fiber created by heating it in an air/acetylene flame and manually pulling the stripped end. The tapered fibers were much more efficient at coupling the evanescent field to the polymer and gave greatly improved results.

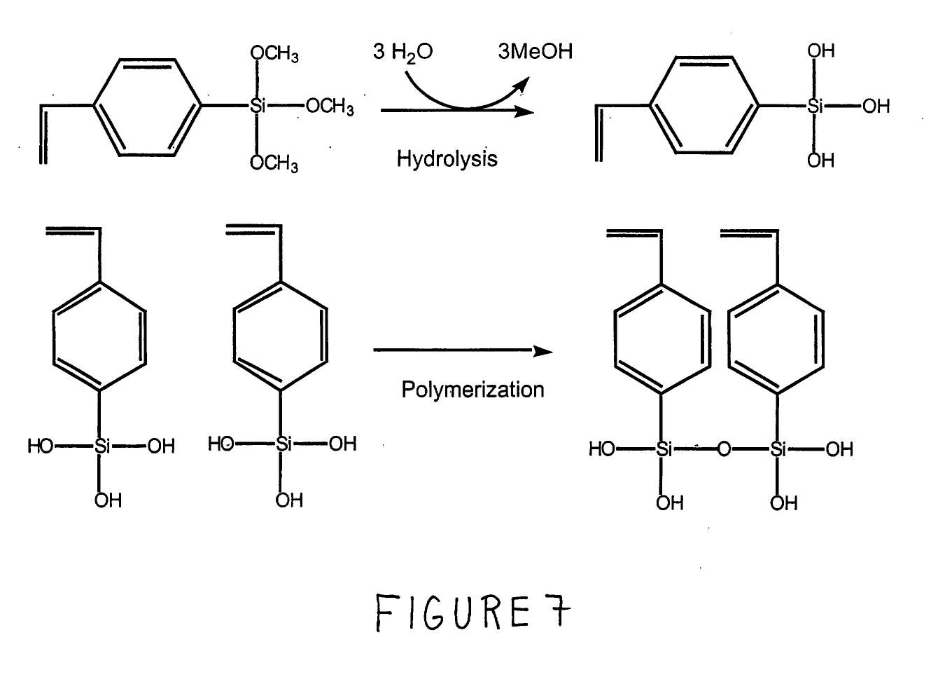

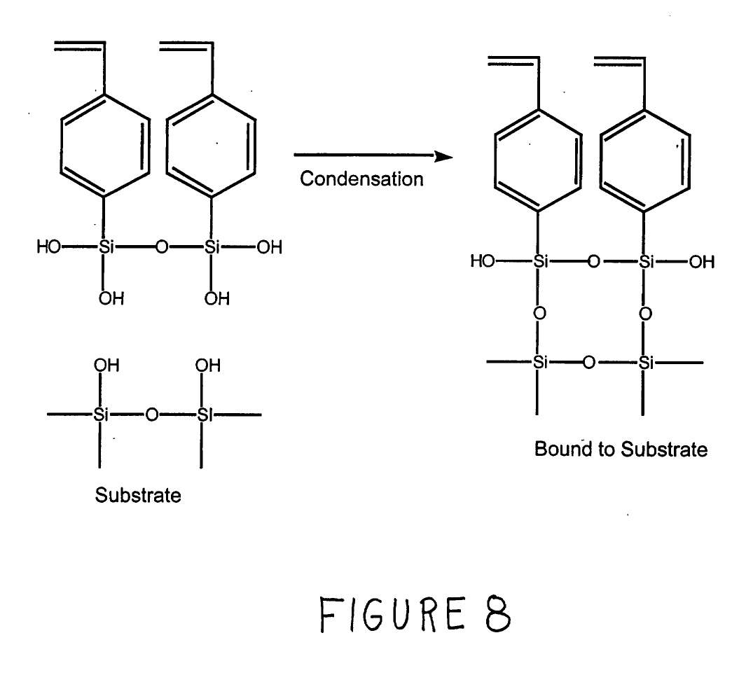

A procedure for preparing optical substrates for polymer coating was developed for the sensor coatings. A solution of trimethoxystyryl silane in toluene is used to place a chemically bound styrene coating on the surface of optical substrates. Experiments were first performed on microscope slides and have shown that the optimal period of silanization using trimethoxystyryl silane in toluene is about twenty minutes. The surface coverage of the layer is indicated by the contact angle made by a drop of water on the surface of the glass. Dip coating of surfaces prepared by silanization (see FIGS. 7 and 8) result in films that are chemically bound to the surface.

### Example 4

### Interferents

The compounds that are most chemically analogous to narcotics are other similar drugs. Many of these compounds exist as liquids, oils or solids at ambient temperatures. Several common chemicals, along with those most chemically similar to were tested using the sensor in order to determine the degree of interference from each narcotic.

Although specific features of the invention are shown in some drawings and not others, this is for convenience only as each feature may be combined with any or all of the other features in accordance with the invention. It will be appreciated that variations thereof can be readily perceived by those skilled in the art, which variations are nevertheless within the scope of the invention as defined by the following claims.

What is claimed is:

1. A fiber optic sensor device for detecting the presence of a narcotic in a fluid, the sensor comprising:
at least one optical fiber means having a proximal end and a distal end for transmitting light energy, the proximal end being disposed within a probe housing;
a molecularly imprinted polymer containing a lanthanide-complex disposed on the distal end of the optical fiber means, wherein the lanthanide-complex is capable of chemically binding with said narcotic;
light source means for generating excitation energy, said light source means operatively associated with said optical fiber means such that said excitation light passes through said optical fiber means; and
detection means operatively associated with said optical fiber means, for detecting an emission signal generated by said lanthanide complex.

2. The device of claim 1 wherein the light source means is selected from the group consisting of an argon laser, blue laser, tunable laser, and light emitting diode.

3. The device of claim 1 wherein the detection means is selected from the group consisting of a spectrophotometer, spectrometer (gas or mass), photomultiplier tube, monochromator equipped with a CCD camera, filters, and the naked eye.

4. The device of claim 1 wherein the lanthanide is selected from the group consisting of: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

5. The device of claim 4 wherein the lanthanide is europium or terbium.

6. The device of claim 1 wherein the distal end of the sensor is enclosed within a semi-permeable membrane.

7. The device of claim 1 wherein the narcotic is selected from the group consisting of amphetamines/methamphetamine, barbiturates, benzodiazepine, buprenorphine, cannabinoids, cocaine, fentanyl, LSD, methadone, nicotine, opiates, morphine, heroin, phencyclidine, metabolites, salts, degradants and pro-drugs thereof.

8. The device of claim 1 wherein the fluid is a vapor.

9. The device of claim 1 wherein the fluid is a liquid.

10. The device of claim 1 wherein the liquid is selected from the group consisting of semen, blood, urine and perspiration.

11. A surface acoustic wave sensor device for detecting the presence of a narcotic in a fluid comprising:
a film of a molecularly imprinted polymer containing a lanthanide-complex disposed on a substrate selected from the group consisting of alumina and quartz crystal;
input and output transducers disposed on the film or substrate; and
a function generator operatively associated with the input transducer for generating a surface acoustic wave along a delay line.

12. The device of claim 11 wherein the lanthanide is selected from the group consisting of: lanthanum (La), cerium (Ce), praseodymium (Pr), neodymium (Nd), promethium (Pm), samnarium (Sm), europium (Eu), gadolinium (Gd), terbium (Tb), dysprosium (Dy), holmium (Ho), erbium (Er), thulium (Tm), ytterbium (Yb), and lutetium (Lu).

13. The device of claim 11 wherein the narcotic is selected from the group consisting of amphetamines/methamphetamine, barbiturates, benzodiazepine, buprenorphine, cannabinoids, cocaine, fentanyl, LSD, methadone, nicotine, opiates morphine, heroin, phencyclidine, and metabolites, salts, degradants and pro-drugs thereof.

14. The device of claim 11 wherein the fluid is a vapor.

15. The device of claim 11 wherein the fluid is a liquid.

16. The device of claim 15 wherein the liquid is selected from the group consisting of semen, blood, urine and perspiration.

17. A method for detecting the presence of a narcotic in a fluid comprising the steps of:
providing a fiber optic sensor device comprising:
at least one optical fiber means having a proximal end and a distal end for transmitting light energy, the proximal end being disposed within a probe housing;
a molecularly imprinted polymer containing a lanthanide-complex disposed on the distal end of the optical fiber means, wherein the lanthanide-complex is capable of chemically binding with said narcotic;
light source means for generating excitation energy, said light source means operatively associated with said optical fiber means such that said excitation light passes through said optical fiber means; and
detection means operatively associated with said optical fiber means, for detecting an emission signal generated by said lanthanide complex; and
exposing at least a portion of said sensor device to an environment containing the fluid such that said narcotic in the fluid binds to the lanthanide-complex in the molecularly imprinted polymer thereby causing the lanthanide-complex to luminesce.

18. The method of claim 17 wherein the lanthanide is selected from the group consisting of: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

19. The method of claim 18 wherein the lanthanide is europium or terbium.

20. The method of claim 17 wherein the narcotic is selected from the group consisting of amphetamines/methampphetamine, barbiturates, benzodiazepine, buprenorphine, cannabinoids, cocaine, fentanyl, LSD, methadone, nicotine, opiates, morphine, heroin, phencyclidine, metabolites, salts, degradants and pro-drugs thereof.

21. The method of claim 17 wherein the fluid is a vapor.

22. The method of claim 17 wherein the fluid is a liquid.

23. The method of claim 22 wherein the liquid is selected from the group consisting of semen, blood, urine and perspiration.

24. A method for detecting the presence of a narcotic in a fluid comprising the steps of:
providing a surface acoustic wave sensor device comprising:
a film of a molecularly imprinted polymer containing a lanthanide-complex disposed on a substrate selected from the group consisting of alumina and quartz crystal;
input and output transducers disposed on the film or substrate; and
a function generator operatively associated with the input transducer for generating a surface acoustic wave along a delay line; and
exposing at least a portion of said sensor device to an environment containing the fluid such that said narcotic in the fluid binds to the lanthanide-complex in the molecularly imprinted polymer thereby causing the lanthanide-complex to luminesce.

25. The method of claim 24 wherein the lanthanide is selected from the group consisting of: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

26. The method of claim 24 wherein the narcotic is selected from the group consisting of amphetamines/methamphetamine, barbiturates, benzodiazepine, buprenorphine, cannabinoids, cocaine, fentanyl, LSD, methadone, nicotine, opiates morphine, heroin, phencyclidine, and metabolites, salts, degradants and pro-drugs thereof.

27. The method of claim 24 wherein the fluid is a vapor.

28. The method of claim 24 wherein the fluid is a liquid.

29. The device of claim 28 wherein the liquid is selected from the group consisting of semen, blood, urine and perspiration.
